(19) United States
(12) Patent Application Publication
(10) Pub. No.: US 2004/0060809 A1
(43) Pub. Date: Apr. 1, 2004

(54) Method for the production of phthalic anhydride to a specification

(76) Inventors: Walter Disteldorf, Wachenheim (DE); Jarren Peters, Mannheim (DE); Bernd Morsbach, Ludwigshafen (DE); Matthias Kummer, Weisenheim (DE); Thomas Ruhl, Frankenthal (DE)

Correspondence Address: KEIL & WEINKAUF, 1350 CONNECTICUT AVENUE, N.W., WASHINGTON, DC, 20036, US

(21) Appl. No.: 10/467,236

(22) PCT Filed: Feb. 6, 2002
(86) PCT No.: PCT/EP02/01196

(30) Foreign Application Priority Data
Feb. 12, 2001 (DE) 10106664.3

(51) Int. Cl. B01D 3/42; B01D 3/00; B01D 3/10; C07D 307/89

(52) U.S. Cl. 203/1; 203/91; 203/99; 203/DIG. 19; 549/250

(57) ABSTRACT

The invention relates to a method for the production of phthalic anhydride to a specification, using a distillative purification of crude phthalic anhydride, by introduction of crude phthalic anhydride into a distillation column, operating at reduced pressure, withdrawal of the low-boiling fraction from the head, or near the head of the column and withdrawal of the phthalic anhydride, which meets the specification, from a side-tap on the column, whilst operating the column at a reflux ratio x, with 1:1.7<x<1:3.

The present invention relates to a process for the preparation of on-spec phthalic anhydride by distillative purification of crude phthalic anhydride, where crude phthalic anhydride is passed to a distillation column which is operated at reduced pressure, the low-boiling components are removed at the top or in the vicinity of the top of the distillation column and the on-spec phthalic anhydride is removed from the column via a side take-off.

Phthalic anhydride is an important basic chemical in the chemical industry. It is used to a considerable extent as a starting material for dialkyl phthalates, which are used in large amounts as plasticizers for plastics such as PVC. Crude phthalic anhydride is prepared industrially from naphthalene and/or o-xylene by catalytic oxidation in the gas phase. For the abovementioned purposes, preference is given to using a phthalic anhydride which has been prepared from o-xylene. The discharges of said customary preparation processes contain, based on their total weight, usually more than 99.5% by weight of phthalic anhydride. The phthalic anhydride is isolated in most cases in liquid form or as a solid using separators.

Depending on the chosen preparation process and particularly on the starting materials and the catalysts, the product contains a spectrum, specific in each case, of impurities and secondary products (cf. e.g.: H. Suter: “Wissenschaftliche Forschungsberichte: II. Anwendungstechnik und angewandte Wissenschaft”, Dr. Dietrich Steinkopff Verlag, Darmstadt, 1972, page 39 ff.; abbreviated below to “Suter”).

On the market, a phthalic anhydride grade with the following specification limits is expected:

solidification point (°C.) min. 130.8

mass fractions (% by weight):

phthalic anhydride min. 99.8

maleic anhydride max. 0.05

benzoic acid max. 0.10 or

max. 0.002 in the case of fragrance quality

phthalic acid max. 0.1

melt color number (Hazen) max. 20

heat color number (Hazen) max. 40

In the art, over the period during which phthalic anhydride has been prepared on an industrial scale, it has become established practice to separate off the secondary products by distillation (cf. e.g.: “Ullmann's Encyclopedia of Industrial Chemistry”, 5th Edition, Vol. A20, VCH Verlagsgesellschaft mbH, Weinheim, 1992, pages 181 to 189; abbreviated below to “Ullmann”; Kirk-Othmer “Encyclopedia of Chemical Technology”, 4th Edition, Vol. 18, John Wiley & Sons, New York, 1996, pages 997 to 1006, abbreviated below to “Kirk-Othmer”). However, impurities which are low-boiling and/or distilazeotropically, some of which have an intense intrinsic color, cause great problems for the person skilled in the art, despite being present in comparatively small amounts.

The distillation—especially its continuous operation frequently of particular interest for cost reasons—is usually carried out by means of two columns in order to obtain a sufficiently pure phthalic anhydride. In the first step, the low-boiling components (for example the greater parts of benzoic acid, maleic anhydride, citraconic anhydride), i.e. substances with a boiling point below the boiling point of the phthalic anhydride, are generally separated off; in a second step, phthalic anhydride is then distilled off from the high-boiling components (for example phthalic acid, certain color-imparting components, condensation products of ingredients of the crude phthalic anhydride), i.e. substances with higher boiling points than that of phthalic anhydride or of undistillable constituents.

In “Suter” (loc. cit., page 45) reference is made to a single-stage continuous distillation of phthalic anhydride (Ruhröl, Europa-Chemie 21, 7 (1965)), but no further details are given.

Particularly high requirements are placed on those esters of phthalic acid synthesized from phthalic anhydride which are to be used as solvents or extenders in perfumes or cosmetics. However, the presence of small amounts of maleic acid, citraconic acid and anhydrides thereof and especially of benzoic acid in phthalic anhydride leads to esterification products of these substances with intense characteristic odor notes, for example a diffuse fruity note in the case of ethyl benzoate. Such impurities are usually to be removed following the ester synthesis by means of a combined washing and extraction step. However, this process is very complex, but, on the other hand, normally makes the preceding customary distillation of the crude phthalic anhydride indispensable.

The problem which arises from the impurities of the phthalic anhydride cannot, on the other hand, be solved satisfactorily with the distillative process disclosed in the earlier International patent application with the file reference PCT/EP/00/07759. There, on-spec phthalic anhydride is obtained by distillative purification of crude phthalic anhydride by passing crude phthalic anhydride to a distillation column which is operated at reduced pressure, and removing the low-boiling components from the column at the top or in the vicinity of the top of the distillation column and removing the on-spec phthalic anhydride from the column via a side take-off.

To reduce the concentration of said undesired accompanying substances, a combined washing and extraction step has instead hitherto usually been required.

It is an object of the present invention to find a technically simple and thus economical process with which crude phthalic anhydride can be purified such that the low contents, demanded on the market, of such secondary components, in particular benzoic acid, which form odor-intensive products upon further reaction of phthalic anhydride to give esters. of phthalic acid, are achieved.

We have found that this object is achieved by a process for the preparation of on-spec phthalic anhydride by distillative purification of crude phthalic anhydride, where crude phthalic anhydride is passed to a distillation column which is operated at reduced pressure, the low-boiling components are removed at the top or in the vicinity of the top of the distillation column and the on-spec phthalic anhydride is removed from the column via a side take-off, which comprises operating the column at a reflux ratio x where 1:1.7<x<1:3.

In the process according to the invention, it is possible to prepare a phthalic anhydride with a content of benzoic acid of less than 20 ppm, preferably between 5 and less than 20 ppm, and also a melt color number of less than 10 APHA and a heat color number of less than 20 APHA.

Suitable distillation columns (also abbreviated below to “columns”) for the purposes of the present invention are tray columns, columns containing dumped packings and columns containing stacked packings, and columns in which the technical features of these column types have been combined. Preference is given to using tray columns. With said column types, it is possible to use customary internals, such as commercial trays, dumped packings or stacked packings, for example bubble trays, tunnel trays, valve trays, sieve trays, dual-flow trays and lattice trays, Pall-Ringe®, Berl® saddles, wire mesh rings, Raschig-Ringe®, Intalox® saddles, Interpak® dumped packings and Intos®, but also stacked packings, such as, for example, Sulzer-Mellapak®, Sulzer-Optiflow®, Kühni-Rombopak® and Montz-Pak®, and fabric packings. In the region below the column feed, internals which are also suitable for solids, in particular dual-flow trays, are preferably chosen. Trays and dumped packings of the abovementioned designs are usually suitable for this purpose.

The column is usually equipped with an evaporator at the bottom and with a condenser at the top of the column.

The diameter of the column depends on the throughputs desired in each case and can be readily determined by a person skilled in the art according to the conventional rules of the art.

The height of the column and the positions of feed and side take-off can be determined using the concept of theoretical plates in conjunction with the internals chosen.

A theoretical plate is defined as that column unit which effects concentration of the readily volatile component according to the thermodynamic equilibrium, assuming ideal mixing, that both phases are in equilibrium and that there is no entrainment of liquid drops-(cf. Vauck, Müller: Grundoperationen chemischer Verfahrenstechnik, VCH Verlagsgesellschaft mbH, Weinheim, 1988).

In general, the column according to the invention is divided into three sections which are determined by the positions of feed, side take-off, top and bottom. The number of theoretical plates for the two upper sections is determined according to the usual process engineering considerations depending on the proportion of low-boiling components in the crude phthalic anhydride and the desired residual content of low-boiling components in the purified phthalic anhydride. The number of theoretical plates for the lower section of the column according to the invention is generally 1 to 10, preferably 2 to 8 and especially 3 to 7.

The process according to the invention is particularly suitable for crude phthalic anhydride as obtained by catalytic gas-phase oxidation of o-xylene and preferably containing more than 95% by weight and in particular more than 98% by weight of phthalic anhydride.

The column is preferably operated at an absolute pressure at the top of the column of from 0.05 to 0.5, preferably 0.1 to 0.3, particularly preferably at 0.15 to 0.25 bar, and a reflux ratio x of preferably 1:1.75 <x<1:2.4 and particularly preferably of 1:1.8 <x<1:2. The temperatures in the column at the top of the column are generally 190 to 220° C., preferably 196 to 202° C. and especially 198 to 200° C., and at the bottom of the column are 220 to 260° C., preferably 230 to 250° C. and especially 235 to 245° C. The temperature at the side take-off is generally 190 to 250° C. and preferably 220 to 240° C.

In one embodiment of the process according to the invention which is in itself particularly preferred, the reflux ratio is 1:1.75 <x<1:2, the temperature at the top of the column is 196 to 202° C., at the bottom of the column is 235 to 245° C. and at the side take-off is 220 to 240° C.

The distillation can be carried out batchwise and, preferably, continuously. The crude phthalic anhydride is passed to the column preferably in gaseous form or particularly preferably in liquid form. Removal in liquid form usually takes place above the column feed, and removal in gaseous form, which is preferred, normally takes place below the feed.

In a preferred embodiment of the process according to the invention where tray columns are used, technical devices such as drop separators can be mounted at the side take-off inside or outside of the column.

The evaporator at the bottom of the column is preferably designed as a falling-film evaporator. Falling-film evaporators are generally known in process engineering. They have the advantages of a lower average residence time of the liquid in the evaporation region, resulting in a more gentle evaporation compared with forced-circulation flash evaporators. As a result of the gentler treatment, the tendency toward the formation of solids can be reduced, the short residence times lead to a reduction in undesired secondary reactions and thus to an improved yield in the bottom, and the operating costs can generally be reduced as a result.

The on-spec phthalic anhydride is usually cooled directly after removal from the column and is obtained as liquid or, after solidification, as a solid. A still higher degree of purity can be achieved, if desired, by subjecting the phthalic anhydride to precision distillation, for example via a side column, or by mounting a dividing wall axially above a certain region in the column (Petlyuk arrangement). Recrystallization is also suitable here.

Via the side take-off, the on-spec phthalic anhydride is normally removed in an amount of at least 97% by weight, based on the weight of the feed. If the column is operated continuously, the amount removed is preferably at least 90% by weight, particularly preferably at least 95% by weight, based on the weight of the feed.

The recovery rate of phthalic anhydride, based on the content in the feed stream to the column, is usually 98% or higher.

The low-boiling components which form during the distillation, and high-boiling components are usually incinerated.

It goes without saying that the process according to the invention can also be optimally adapted for individual separation problems. This adaptation can be carried out routinely by the person skilled in the art if he follows the teaching disclosed herein.

The purity of the resulting phthalic anhydride can be determined by generally known analytical methods, such as gas chromatography, UV spectroscopy and acid-base titration. Since a phthalic anhydride without coloring impurities is required for most use purposes, particular importance is attached to characterization by color numbers—particularly the melt color number and the heat color number. Color changes in the phthalic anhydride under thermal stress are of practical importance since phthalic anhydride is normally stored and transported in the molten state—for example at 160° C. In particular, the melt color number (APHA/Hazen color scale, cf. W. Liekmeier, D. Thybusch: Charakterisierung der Farbe von klaren Flüssigkeiten, Editor: Bodenseewerk Perkin-Elmer GmbH, Überlingen, 1991) is generally ascertained by determining the color number of phthalic anhydride immediately after sampling at a temperature of 160° C. Furthermore, the heat color number is generally ascertained by keeping the phthalic anhydride at 250° C. for 90 minutes and then measuring the color number.

Determination of the content of benzoic acid in phthalic anhydride is possible by means of conventional quantitative gas chromatography.

Using the process according to the invention, the content of the abovementioned undesired readily volatile accompanying substances, primarily benzoic acid, in the phthalic anhydride can be reduced to values of less than 20 ppm by weight, such that the resulting phthalic anhydride then satisfies even the most stringent requirements of the fragrance and cosmetics industry.

### EXAMPLE

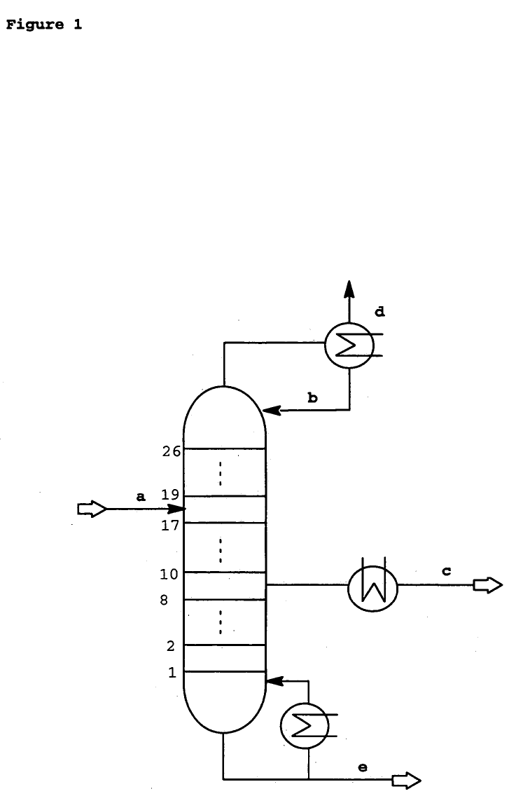

A tray column according to the diagrammatic FIG. 1 was used. The column had 26 valve trays (approximately 16 theoretical plates) and had a diameter of 50 mm. The side take-off was located between the eighth and the tenth tray above the bottom (roughly in the region between the fifth and the sixth theoretical plate), and the feed was located between the 17th and the 19th tray above the bottom (roughly in the region of the eleventh theoretical plate). In FIG. 1, the 1st and 2nd tray are also shown, and the other trays are indicated by perpendicular dashed lines.

The crude phthalic anhydride distilled was one which had been prepared by gas-phase oxidation of o-xylene over a fixed bed in the presence of a catalyst consisting of a support core coated with the catalytically active metal oxides cesium oxide (calculated as 0.4% by weight of cesium), vanadium oxide (4% by weight) and titanium dioxide (95.6% by weight) (cf. the earlier German patent application with the file reference 198 24 532). The loading in the reactor was 86 g of o-xylene per m³ (S.T.P.) of air. The reactor temperature was between 350 and 450° C.

The resulting crude phthalic anhydride had the following weight-based composition:

| | |
|---|---|
| 99.24% by weight | of phthalic anhydride |
| 0.2% by weight | of benzoic acid |
| 200 ppm | of maleic anhydride |
| 20 ppm | of citraconic anhydride |
| 0.5% by weight | of phthalic acid |

and the remainder to 100% by weight of other substances.

1000 g/h of this crude phthalic anhydride were passed continuously (a) to the column. At a reflux of 530 g (b), an absolute pressure of 0.17 bar at the top of the column, a temperature. of 198° C. at the top of the column and 238° C. at the bottom of the column, 970 g of purified phthalic anhydride were removed within the same time via the side take-off at 221° C., condensed and isolated (c). The top take-off via (d) was condensed into a cold trap and amounted to approximately 7 g; the bottom take-off via (e) amounted to approximately 15 g and contained the high-boiling and nondistillable components. Analysis of the phthalic anhydride isolated via the side take-off at (c) gave the following weight-based composition:

| | |
|---|---|
| 99.97% by weight | of phthalic anhydride |
| 15 ppm | of benzoic acid |
| <10 ppm | of maleic anhydride |
| <10 ppm | of citraconic anhydride |
| 0.02% by weight | of phthalic acid |

and the remainder to 100% by weight of other substances.

The melt color number was determined directly following removal and was 5-10 APHA. The heat color number was determined as follows: a sample of phthalic anhydride was heat-treated in a drying oven for 1.5 hours at a temperature of 250° C. The color number was then measured as 10-20 APHA.

We claim:

1. A process for the preparation of on-spec phthalic anhydride by distillative purification of crude phthalic anhydride, where crude phthalic anhydride is passed to a distillation column which is operated at reduced pressure, the low-boiling components are removed at the top or in the vicinity of the top of the distillation column and the on-spec phthalic anhydride is removed from the column via a side take-off, which comprises operating the column at a reflux ratio x where 1:1.7<x<1:3.

2. A process as claimed in claim 1, wherein the distillation is carried out at a pressure at the top of the column of from 0.05 to 0.5 bar.

3. A process as claimed in claim 1 or 2, wherein the side take-off is located closer to the upper of the two trays between which it is arranged.

4. A process as claimed in any of claims 1 to 3, wherein the on-spec phthalic anhydride is removed from the column in gaseous form via the side take-off.
